(19) United States
(12) Patent Application Publication
(10) Pub. No.: US 2016/0022271 A1
(43) Pub. Date: Jan. 28, 2016

(54) FLAT WOUND DETACHABLE EMBOLIZATION COIL

(71) Applicants: DeLois Marlene FERRY, Excelsior, MN (US); Steven J. FERRY, Excelsior, MN (US)

(72) Inventors: Steven J. Ferry, Excelsior, MN (US); DeLois Marlene Ferry, Excelsior, MN (US)

(21) Appl. No.: 14/774,787

(22) PCT Filed: Mar. 10, 2014
(86) PCT No.: PCT/US2014/022590

§ 371 Date: Sep. 11, 2015

Related U.S. Application Data
(60) Provisional application No. 61/776,465, filed on Mar. 11, 2013.

(51) Int. Cl. A61B 17/12

(52) U.S. Cl. CPC A61B 17/1215; A61B 17/12031; A61B 17/12109; A61B 2017/12068; A61B 2017/12086; A61B 2017/1209; A61B 2017/12054; A61B 2017/00836

(57) ABSTRACT

Inventive subject matter disclosed herein includes a coil assembly for treating a vascular malformation, comprising: a flat wire coil comprising Palladium, having an optimum packing density, that is coiled and that is detachable attachable to the flat wire coil.

### CLAIM OF PRIORITY

This application claims the benefit of U.S. Provisional Patent Application Ser. No. 61/776,465, filed on Mar. 11, 2013, the benefit of priority of which is claimed hereby, and which is incorporated by reference herein in its entirety.

### FIELD

Inventive subject matter disclosed herein relates to a flat wire palladium (Pd) embolization coil for occluding malformations within the human vasculature.

### BACKGROUND

The prior art includes methods for occluding vessel openings. However, the methods are catheter based and employ a very thin wall compliant balloon on a distal tip of the catheter to achieve occlusion. These devices also require the use of a guidewire to track to a desired location. In addition, because of the thin wall, when the distal balloon is inflated with contrast media, the balloon is susceptible to rupture as well as being susceptible to rupture in use.

### SUMMARY

Inventive subject matter disclosed herein includes a coil assembly for treating a vascular malformation. The coil assembly includes a flat wire coil comprising Palladium and or Platinum in a thickness and width ranging from 0.0005 inches by 0.003 inches coiled to a pitch of 0.000 to 0.012 inches, a cut width in a range 0.0001 to 0.005 inches and a length of 10 cm to 90 cm, having an inner diameter in a range from of 0.004 to 0.0016 inches and an outer diameter within a range of 0.006 to 0.018 inches. The coil assembly also includes a detacher that is detachably attachable to the flat wire coil.

Inventive subject matter also includes a coil assembly for treating a vascular malformation. The coil assembly includes a flat wire coil comprising Palladium or other such metal, having an optimum packing density, coiled to a pitch of 0.001 inches, a cut width of 0.0007 inches and a length of 90 cm, having an inner diameter of 0.012 inches and an outer diameter within a range of 0.006 to 0.018 inches. The coil assembly also includes a detacher that is detachably attachable to the flat wire coil.

### DESCRIPTION OF THE DRAWINGS

FIG. 1 illustrates a side view of a coil for treating a vessel malfunction.

FIG. 1A illustrates a side view of another embodiment of a distal coil segment.

FIG. 1B illustrates a side view of a safety tether of FIG. 1.

FIG. 1C illustrates a longitudinal cross-sectional view of a mechanical detachment system for a coil for treating a vessel malfunction.

FIG. 2A illustrates a side cross-sectional view of a mechanical detachment system for the coil of FIG. 1.

FIG. 2B illustrates a side cross-sectional view of a mechanical detachment system for the coil of FIG. 1.

FIG. 3 illustrates a side cross-sectional view of a mechanical detachment system for the coil of FIG. 1.

FIG. 4A illustrates a side cross-sectional view of a magnetic detachment system for the coil of FIG. 1 when magnets have no current passing through them.

FIG. 4B illustrates a side cross-sectional view of a magnetic detachment system for the coil of FIG. 1 when current passes through a tether magnet.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

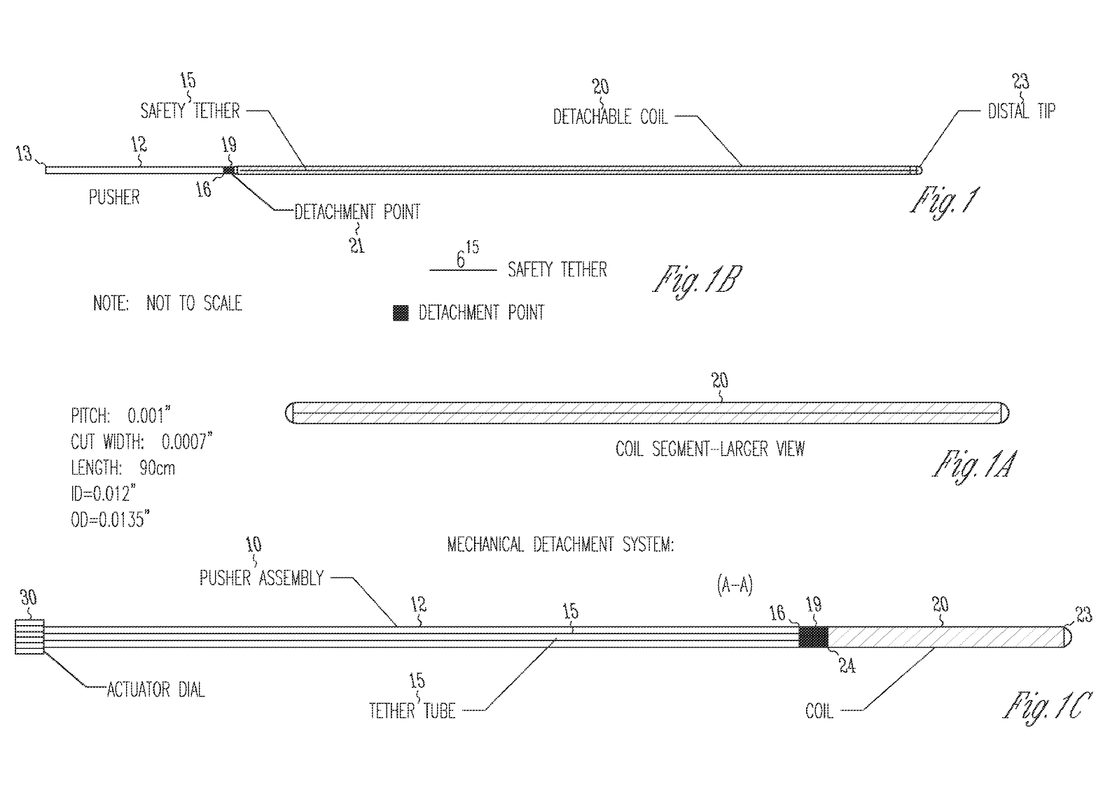

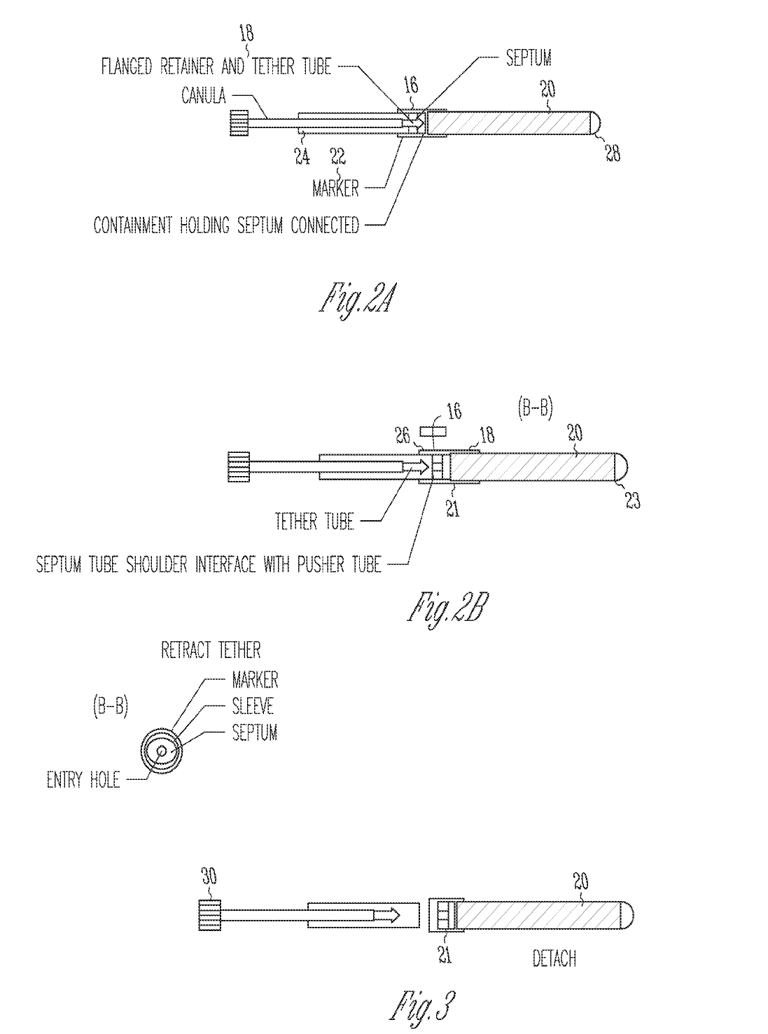

Inventive embodiments disclosed herein include a coil assembly 10 that includes a detachable coil 20 and pusher assembly 12, such as is shown in FIGS. 1 and 1A, effective for use in the treatment of vascular malformations such as, but not limited to, cerebral aneurysms and the like. Said coil assembly 10 has an outer diameter ranging from 0.006″ to 0.018″. The coil assembly 10 includes a distal tip 23 and a proximal end 13.

The distal tip 23 of the coil assembly device 10 is part of the detachable distal coil 20, shown in FIGS. 1 and 1A. The detachable coil 20 is fabricated with flat wire at a specified pitch and gap specification. A safety tether 15 extends from the distal tip 23 to proximal end 21 of the detachable coil 20. The tether 15 prevents the detachable coil 20 from unwinding if it becomes caught. A detachment point 19 is positioned into the proximal end 21 of the coil body 20 and distal end 16 of the pusher 12. For this embodiment, detachment is achieved by conventional inductive heating to melt a polymer tether, or magnetic detachment or mechanical detachment. The mechanical and magnetic systems are discussed herein.

Finally, the pusher assembly 12 is connected to the detachable coil 20 which is used to provide axial force for the delivery of the detachable coil 20 to a desired location in a living being. It is believed that by using the flat wire configuration, the detachable coil 20 is positioned in such a manner while being deployed so as to improve the packing density of the deployed coil as compared to conventional round wire GDC coils.

### Mechanical Detachment:

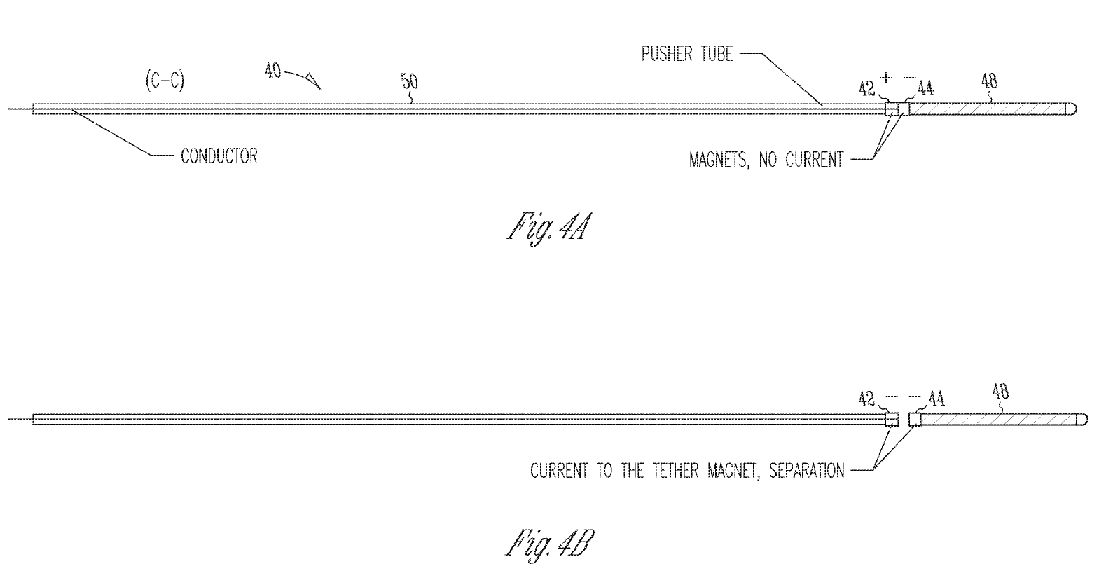

The coil assembly inventive embodiment disclosed herein at 10 in FIG. 1 is configured with the pusher assembly 12 having an outer tube within which a tether tube 15 is placed. On a distal end 16 of the tether tube 15 a flanged retainer 18 with an inner diameter is positioned into the end 16 of the tether tube 15 and affixed in an appropriate manner known in the art. See (A-A) and (B-B) in FIGS. 1C and 2B. The flanged retainer 18 is affixed to the detachable coil body 20, having proximal end 21 and distal end 23. On the proximal end 21 of the detachable coil body 23, is an assembly that includes a cylindrical marker 22 into which a smaller canister 24 with a septum 26 is placed so as to allow the septum 26 to be facing the flanged retainer 18. When the flanged retainer 18 is pushed through the septum 26, a secure joint is formed, as shown in FIG. 2B. To detach the detachable coil body 20, the pusher tube 12 is held and a tether actuator dial 30 is rotated from its locked position and the flanged retainer 18 is withdrawn proximately through the septum 26, thereby releasing the detachable coil 20 from the pusher assembly 12, as shown in FIG. 3. A new coil may now be placed on the coil assembly 10 in the same manner.

In another embodiment, instead of a septum, the flanged retainer tube is bonded into place at the proximal end of the detachable coil, using the appropriate material. Detachment occurs, when the canula tube is used to buttress the detachment point of the coil while the tether tube is withdrawn resulting in separation. Then, the entire pusher assembly is removed from the microcatheter leaving the coil in the desired location. It should be noted that the flanged retainer can be mounted to a catheter or microcatheter or mounted to be integral a solid round wire as well. The illustration provided in FIG. 3 shows a tubular design.

In one embodiment, the detachable coil body has a pitch of 0.001 inches, a cut width/thickness of 0.0007 inches, a length of 90 cm, an inner diameter of 0.012 inches and an outer diameter of 0.0135 inches. The wire was a Pd flat wire, 304V SST flat wire (0.001in. to 0.003 in.) In one embodiment, the wire has rounded edges.

### Magnetic Detachment:

In another embodiment, a pusher assembly illustrated at 40 in FIG. 4 employs a small magnet 42 encapsulated and mounted to a distal end 44 of a tether tube 46. A second sealed magnet 44 is placed within a proximal end of a coil body 48, as shown in FIG. 5. Detachment can be achieved by either employing a current supplied to the tether magnet 42 in order to alter the polarity, whereby matching the same polarity on both magnets (++ or −−) 42 and 44 resulting in repulsion and separation. See (C-C) in FIG. 4. Alternatively, magnet canisters can be assembled in the same manner, however a cylindrical magnet has a (++) half and a (−−) half. When in the (+/−−) orientation between the tether and coil magnets 42 and 44, there is attachment. When the tether/tether magnet 42 is manually rotated 180° resulting in a (++) (−−) of the two halves of the tether magnet relative to the coil magnet 44, repulsion and separation occur. See (D-D) shown in FIG. 5.

### Magnetic Detachment

In another embodiment the coil 48 is detached by incorporation of two small magnets 42 and 44 in the proximal end of the coil 48 and the distal mating end 41 of the pusher tube 50, shown in FIG. 4. Said magnets 42 and 44 are encapsulated in a biocompatible material to ensure biocompatibility and stability. Detachment occurs by applying a current and changing the polarity on the tether side magnet 42, whereby resulting in same polarity and repulsion, thus separation of the coil 48 from the pusher. 50. It should be noted that the magnets 42 and 44 can be mounted onto a solid round wire as well the flat wire disclosed herein.

### System:

Magnetic polarity is changed in the wire magnet mechanically as well. Magnetic polarity is changed by using a magnet tipped tether which runs the length of the pusher and which has an actuator that allows the user to rotate the magnet tipped cannula and changing the polarity of the tether magnet, thus again attaining separation, as shown in FIG. 5 at (D-D).

Rotational direction of actuator dial 60 may be clockwise or could be counter clockwise

### Detachment

Embodiments disclosed herein include a flat wound coil design, wherein coiling is achieved on the flat of the wire or on the edge, both providing different characteristics to the respective coils. The device has an improved packing density as a result of the ribbon like coil. Given the thin thickness of the coil, the coil compresses providing greater capacity for additional material. The device employs flat wire of pure Palladium (Pd), understanding that purity at its best is 99.995 percent in natural ore. Some device embodiments employ aPd/Ti alloy, which has good radiopacity at a lower cost than Pt material. Ti at a fractional concentration.

The flat wire configuration has a greater surface area that conventional round wire. The flat wire may be precoated prior to coiling to improve platelet aggregation.

The mechanical concept for detachment eliminates a need for an inductive detachment system.

Magnetic detachment eliminates a need for an inductive detachment system.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

What is claimed is:

1. A coil assembly for treating a vascular malformation, comprising:
a flat wire coil comprising Palladium and or Platinum coiled to a defined pitch, with a slot width and a length, also, having an inner diameter and an outer diameter, and a detacher, detachably attachable to the flat wire coil.

2. The coil assembly of claim 1, further comprising a safety tether positioned from the distal to proximal end of the flat wire coil for preventing the coil from unwinding.

3. The coil assembly of claim 1, further comprising a pusher tube connected to the flat wire coil effective for providing axial force to the delivery of the coil to a selected location.

4. The coil assembly of claim 1, wherein the detacher includes a heat based detacher, magnetic detacher, or mechanical detacher system.

5. The coil assembly of claim 3, wherein the pusher tube encloses a tether tube that contains the tether onto which a flanged retainer, magnet or conductor wire can be affixed.

6. The coil assembly of claim 3, wherein the pusher tube encloses a solid wire onto which a flanged retainer or magnet are affixed and through which a current could be passed.

7. The coil assembly of claim 1, wherein the flat wire coil includes a precoat.

8. A coil assembly for treating a vascular malformation, comprising:
a flat wire coil comprising Palladium and or Platinum, having an optimum packing density, coiled to a pitch, having an inner diameter an outer diameter, and a (detacher, detachably attachable) to the flat wire coil.
